(19) United States
(12) Patent Application Publication
(10) Pub. No.: US 2021/0307839 A1
(43) Pub. Date: Oct. 7, 2021

(54) SYSTEM AND METHOD FOR MINIMALLY INVASIVE LOWER ESOPHAGEAL SPHINCTER STIMULATION

(71) Applicant: Covidien LP, Mansfield, MA (US)

(72) Inventors: David N. Heard, Boulder, CO (US); Joe D. Sartor, Boulder, CO (US)

(21) Appl. No.: 17/205,204

(22) Filed: Mar. 18, 2021

Related U.S. Application Data
(60) Provisional application No. 63/004,008, filed on Apr. 2, 2020.

(51) Int. Cl. A61B 34/20; A61B 90/00; A61N 1/05; A61B 5/06; A61N 1/375; A61N 1/36

(52) U.S. Cl. CPC A61B 34/20; A61B 90/37; A61N 1/0517; A61B 5/062; B06B 1/0292; A61N 1/36007; A61B 2034/2063; A61B 2090/3786; A61N 1/37518

(57) ABSTRACT

A system and method for treating gastroesophageal reflux disease by using an implantation tool to implant a stimulation electrode in a lower esophageal sphincter for stimulation of the lower esophageal sphincter muscle.

### CROSS-REFERENCE TO RELATED APPLICATION

This application claims the benefit of and priority to U.S. Provisional Patent Application No. 63/004,008, filed on Apr. 2, 2020, the entire disclosure of which is incorporated by reference herein.

### FIELD

The present technology is generally related to a system and method for minimally invasive lower esophageal sphincter stimulation, such as for treatment of gastroesophageal reflux disease.

### BACKGROUND

Patients who suffer from sphincter control deficiencies, such as, for example, gastric reflux disease, have sphincters which may not close fully, thereby allowing leakage of gastric acid into portions of the gastrointestinal tract such as the esophagus. In cases regarding gastric reflux disease, gastric acid refluxing into the esophagus may injure the esophageal mucosa and underlying muscle, causing permanent damage to the sphincter which may lead to further loss of barrier function. To counter a poorly functioning sphincter, patients may be prescribed pharmaceutical remedies, such as, for example, proton-pump inhibitors, or may undergo a surgical procedure to assist closure of the sphincter. Such surgical procedures may include, for example, anti-reflux surgery, such as Nissen fundoplication, or implantation of a sphincter assist device which circumscribes the sphincter, such as, for example, a rubber band or a ring of magnetically attracted elements.

Preventing relaxation of the lower esophageal sphincter and/or increasing lower esophageal sphincter tone, in order to increase the barrier action of the lower esophageal sphincter, is known for reducing the exposure of esophageal mucosa to gastric acid reflux. Most treatment techniques rely on inducing scarring and/or hypertrophy of the lower esophageal sphincter to reduce, or prevent, relaxation of the lower esophageal sphincter by producing injury using radio frequency or thermal ablation. Other known procedures attempt to alter the lower esophageal sphincter by placing mucosal sutures or submucosal injection of silicone in the lower esophageal sphincter. However, all of these methods suffer from the major disadvantage in that they induce injury. Further, the results are substantially irreversible, and there is limited ability to make adjustments without requiring subsequent endoscopic procedures.

### SUMMARY

This disclosure generally relates to a system and method for a minimally invasive procedure for implanting a device configured to stimulate the lower esophageal sphincter. The disclosed method is reversible and avoids laparoscopic electrode placement.

In one aspect, a system includes an implantation tool, a stimulation electrode configured to stimulate the lower esophageal sphincter muscle to treat gastroesophageal reflux disease, and a display. The implantation tool is navigable through an incision to a lower esophageal sphincter muscle and the display is configured to display ultrasound images generated by an ultrasound sensor of the implantation tool. The implantation tool includes a handle configured to be grasped by a user, a cannula extending distally from the handle and defining a lumen therethrough, an elongated rod removably positioned through the lumen of the cannula, and an ultrasound sensor disposed at a distal portion of the elongated rod and oriented to provide a forward-looking perspective of ultrasound images of objects distally of the elongated rod.

In an aspect, the system further includes a tracking system configured to track a position of the implantation tool. The tracking system may be an electromagnetic tracking system configured to generate an electromagnetic field and the implantation tool may include a position sensor configured to sense the electromagnetic field.

In an aspect, the implantation tool includes a position reference guide extending from the handle and configured to provide extracorporeal positional and directional reference for which way the rod and cannula are pointing within the body during navigation of the implantation tool.

In an aspect, the stimulation electrode is configured to removably secure to a lower esophageal sphincter muscle. The stimulation electrode may include a helical tip at its distal portion to screw the helical tip of the stimulation electrode into the lower esophageal sphincter muscle upon rotation of the stimulation electrode.

In an aspect, the cannula is removable from the implantation tool such that the elongated rod is removable from the cannula after a distal portion of the cannula is navigated proximate a lower esophageal sphincter muscle to provide access for placement of a stimulation electrode through the cannula.

In an aspect, the ultrasound sensor is a capacitive micromachined ultrasonic transduce.

In an aspect, the system further includes an ultrasound imaging device, separate from the ultrasound sensor of the implantation tool, the ultrasound imaging device configured to acquire ultrasound data externally from a patient.

In another aspect, an implantation tool is configured to navigate through a small incision to a lower esophageal sphincter muscle. The implantation tool includes a handle configured to be grasped by a user, a cannula extending distally from the handle and defining a lumen therethrough, an elongated rod removably positioned through the lumen of the cannula, and an ultrasound sensor disposed at a distal portion of the elongated rod and oriented to provide a forward-looking perspective of ultrasound images of objects distally of the elongated rod.

In an aspect, the implantation tool includes a position sensor configured to operably couple to a tracking system for tracking a position of the implantation tool during navigation.

In an aspect, the stimulation electrode is configured to removably secure to a lower esophageal sphincter muscle and configured to stimulate the lower esophageal sphincter muscle to treat gastroesophageal reflux disease. The stimulation electrode may include a helical tip at its distal portion to screw the helical tip of the stimulation electrode into the lower esophageal sphincter muscle upon rotation of the stimulation electrode.

In an aspect, the cannula is removable from the implantation tool such that the elongated rod is removable from the cannula after a distal portion of the cannula is navigated proximate a lower esophageal sphincter muscle to provide access for placement of a stimulation electrode through the cannula.

In an aspect, the ultrasound sensor is a capacitive micromachined ultrasonic transducer.

In an aspect, the implantation tool includes a position reference guide extending from the handle and configured to provide extracorporeal positional and directional reference for which way the rod and cannula are pointing within the body during navigation of the implantation tool.

In another aspect, a method for treating gastroesophageal reflux disease includes inserting an elongated rod surrounded by a cannula through an abdominal incision, navigating the elongated rod and cannula to a lower esophageal sphincter muscle, displaying front view ultrasound images during the navigating, the front view ultrasound images generated by an ultrasound sensor coupled to a distal portion of the elongated rod, implanting a stimulation electrode into the lower esophageal sphincter muscle, and stimulating the lower esophageal sphincter muscle by the stimulation electrode to treat the gastroesophageal reflux disease.

In an aspect, the method further includes utilizing an electromagnetic tracking system to track a position of the elongated rod during the navigating.

In an aspect, the method further includes screwing a helical tip of the stimulation electrode into the lower esophageal sphincter muscle by rotating the stimulation electrode prior to stimulating the lower esophageal sphincter muscle.

In an aspect, the method further includes displaying secondary ultrasound data during the navigating of the elongated rod.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram of a system for implanting a device configured to stimulate a lower esophageal sphincter in accordance with an aspect of the disclosure;

FIG. 2 is a side perspective view of an implantation tool of the system of FIG. 1;

FIG. 3 is a side perspective view of a stimulation electrode usable with the implantation tool of FIG. 2; and

FIG. 4 is a flow chart illustrating a method of implanting the stimulation electrode of FIG. 3 adjacent a lower esophageal sphincter to treat gastroesophageal reflux disease in accordance with an aspect of the disclosure.

### DETAILED DESCRIPTION

This disclosure generally relates to a system and method for a minimally invasive procedure for implanting a device configured to stimulate the lower esophageal sphincter for treatment of gastroesophageal reflux disease. The disclosed method is reversible and avoids laparoscopic electrode placement.

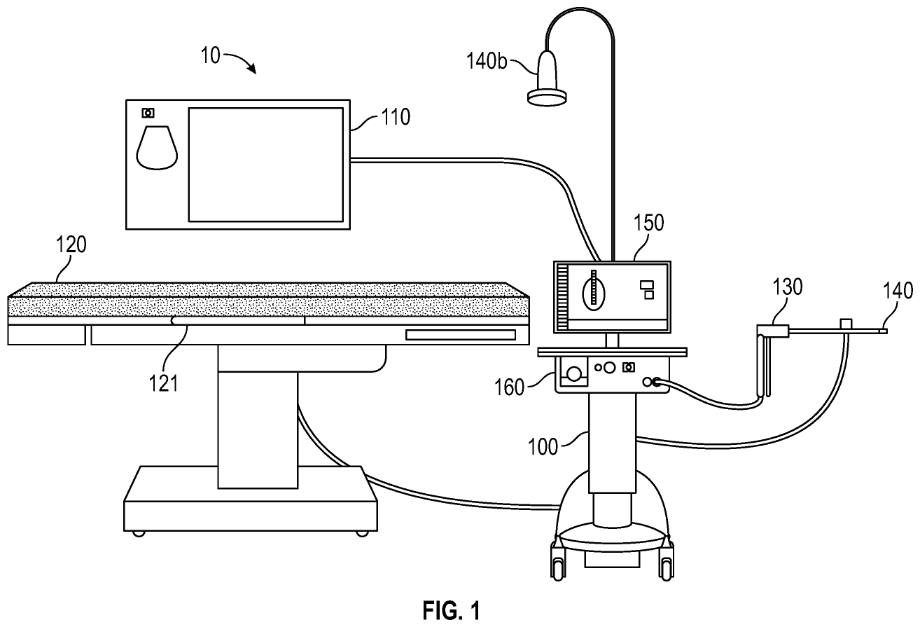

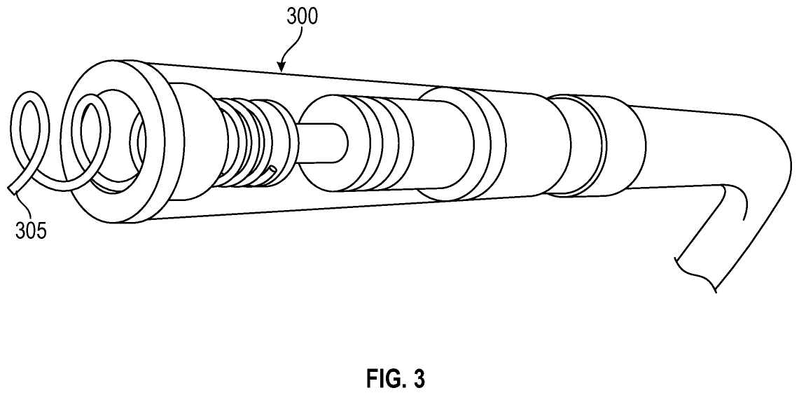

FIG. 1 illustrates a system 10 for implanting stimulation electrode 300 (FIG. 3) configured to stimulate the lower esophageal sphincter. The system 10 includes a computing device 100, a display 110, a table 120, an implantation tool 130, an ultrasound workstation 150, and an optional electrosurgical generator 160 for assisting with surgical procedure. The implantation tool 130 is navigable through a small incision into the abdomen of a patient to position a cannula 133, which provides access for placement of the stimulation electrode 300 at the lower esophageal sphincter for stimulation to treat gastroesophageal reflux disease.

The computing device 100 may be, for example, a laptop computer, desktop computer, tablet computer, or other similar device. The computing device 100 may be configured to control an electrosurgical generator, a power supply, and/or any other accessories and peripheral devices relating to, or forming part of, the system 10.

The display 110 is configured to output instructions, images, and messages relating to the performance of the stimulation procedure in the form of the graphical user interfaces. Although the display 110 is shown as a separate component from the computing device 100, in aspects, the display 110 is a component of the computing device 100, where the computing device 100 includes one or more displays for displaying various user interfaces displaying data corresponding to ultrasound data and navigation and stimulation parameters and data. The table 120 may be, for example, an operating table or other table suitable for use during a surgical procedure, which includes an electromagnetic (EM) field generator 121. The EM field generator 121 is used to generate an EM field during the stimulation procedure and forms part of an EM tracking system which is used to track the positions of surgical instruments, such as the implantation tool 130, within and around the body of a patient. The EM tracking system (or another tracking system) may additionally include sensors for tracking movement of the patient (e.g., breathing) and may utilize such patient tracking movement to compensate for any displayed elements. Such sensors may include one or more electromagnetic tracking sensors positionable on a patient's chest, which tracks patient body movement independently from any other device (e.g., implantation tool 130) movements.

The location of the implantation tool 130 within the body of the patient may be tracked during the surgical procedure and navigation to the lower esophageal sphincter. An exemplary method of tracking the location of the implantation tool 130 may be performed by using the EM tracking system, which tracks the location of the implantation tool 130 through tracking sensors (e.g., position sensor 135) attached to or incorporated in the implantation tool 130.

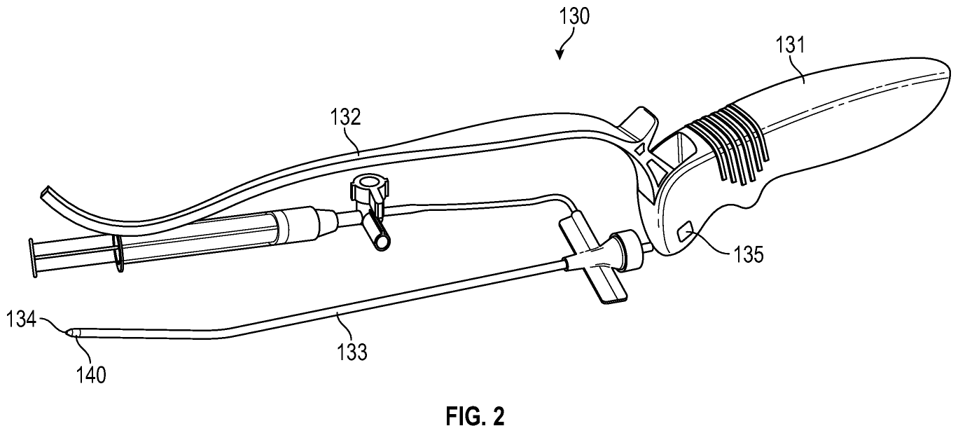

FIG. 2 illustrates the implantation tool 130 which includes a handle 131, a position reference guide 132, an elongated rod 134, and an ultrasound sensor 140. In aspects, the implantation tool 130 may also include a position sensor 135 for tracking the position of the implantation tool 130 or its components, and a cannula 133 through which the elongated rod 134 is removably positioned. The elongated rod 134 is configured to slidably move within and through the cannula 133. The elongated rod 134 may also be rotated within the cannula 133.

The cannula 133 is useful for providing access to the lower esophageal sphincter after a distal end of the cannula 133 is properly positioned relative to the lower esophageal sphincter using the elongated rod 134. A distal end of the cannula 133 may include a sharpened edge for cutting tissue during navigation, or a blunt edge for retracting tissue during navigation. When a cannula 133 is used with the implantation tool 130, the elongated rod 134 is removably positioned through a lumen of the cannula 133. During use, once the cannula 133 is properly positioned (e.g., the distal end of the cannula 133 is navigated proximate, or in front of the lower esophageal sphincter), the implantation tool 130 may be separated from the cannula 133 and removed from the patient while the cannula 133 is positioned in place, with the distal end of the cannula 133 facing the lower esophageal sphincter. With this configuration, the cannula 133 provides access for other tools to be placed through its lumen for access to the lower esophageal sphincter. For example, the cannula 133 may be used to provide access for inserting the stimulation electrode 300 (FIG. 3) to the lower esophageal sphincter via the elongated rod 134 or another delivery device. In an aspect, the cannula 133 may have an inner diameter of about 3 mm, although other diameters are contemplated. Additionally, the cannula 133, may be used to provide access for a wired connection between the implanted stimulation electrode 300 and an external stimulation generator, for example, an external stimulation generator worn on a patient.

Each of the position reference guide 132 and the elongated rod 134 extends distally from the handle 131. A distal end of the elongated rod 134 is positionable through a small incision of a patient and into the abdomen of the patient. The position reference guide 132 is movable relative to the handle 131 and is used to provide extracorporeal positional and directional reference for which way the elongated rod 134 (and optionally the cannula 133) is pointing within the body while the elongated rod 134 of the implantation tool 130 is navigated to the lower esophageal sphincter.

The elongated rod 134 of the implantation tool 130 is navigable through the small incision into the abdomen, to navigate the cannula 133 to the lower esophageal sphincter, and to place the stimulation electrode 300 at the lower esophageal sphincter for stimulation to treat gastroesophageal reflux disease. A distal end of the elongated rod 134 may include a sharpened edge for cutting tissue during navigation, a blunt edge for retracting tissue during navigation. In an aspect, the elongated rod 134 may have a diameter of about 3 mm, although other diameters are contemplated.

A distal portion of the elongated rod 134 includes a forward-facing ultrasound sensor 140 which provides a forward-looking view of the objects distally, i.e., in front, of the elongated rod 134. The ultrasound sensor 140 may be a capacitive micromachined ultrasonic transducer. The ultrasound sensor 140, and in particular, its forward-looking orientation, is useful in navigating between, and past various organs and tissue during navigation of the elongated rod 134, such as liver and spleen and provides an in-line view which is more intuitive for navigation when compared to other oblique views, such as traditional externally imaged ultrasound transducers. Additionally, the ultrasound sensor 140, or another component of the implantation tool 130 of system 10, for example ultrasound device 140b (FIG. 1), may include doppler or other ultrasound techniques which assist in avoiding blood vessels that are near the esophagus or other anatomy. As described above, images generated from the data acquired by the ultrasound sensor 140 along with images obtained from other ultrasound devices are displayed on the display 110 (FIG. 1) for viewing during the navigation and stimulation procedure.

The elongated rod 134 is configured to operably couple to the stimulation electrode (FIG. 3) for delivering the stimulation electrode 300 to the lower esophageal sphincter. In this configuration, the elongated rod 134 is used to insert the stimulation electrode 300 into the lower esophageal sphincter. FIG. 3 illustrates the stimulation electrode 300 which is configured to pass through the cannula 133 after the elongated rod 134 is removed from the cannula 133 for delivery of the stimulation electrode 300 to the lower esophageal sphincter. The stimulation electrode 300 is configured to be secured to the lower esophageal sphincter muscle and be removed therefrom if desired.

The stimulation electrode includes a helical tip 305 at its distal end for securing the stimulation electrode 300 to the lower esophageal sphincter. In one aspect, the stimulation electrode 300 is rotatable to screw the helical tip 305 into tissue (e.g., the lower esophageal sphincter muscle) to secure the stimulation electrode 300 to the tissue. In this manner, the stimulation electrode 30 is secured to the lower esophageal sphincter muscle while also being removable therefrom (by unscrewing the helical tip 305 from the tissue) if removal is desired. Alternatively, the elongated rod 134 may be coupled to the stimulation electrode 300 such that the elongated rod 134 may be used to screw the helical tip 305 of the stimulation electrode 300 into the lower esophageal sphincter or other tissue. The helical tip 305 of the stimulation electrode 300, or another portion of the stimulation electrode 300, is conductive for delivery of the stimulation energy. As the lower esophageal sphincter is subject to much movement, the elongated rod 134 (and optionally, the cannula 133) may be guided through connective or striated muscle tissue in order to support the stimulation electrode 300 in a manner that reduces the recurrent or maximum stress on the conductive portion of the stimulation electrode 300 to prevent fracture of any portion thereof.

The stimulation electrode 300 may be self-powered by an internal stimulation generator or externally powered (e.g., by the elongated rod 134 or a stimulation generator located outside the patient's body) to provide stimulation energy to the lower esophageal sphincter. In an aspect, the stimulation electrode 300 may have a diameter of about 3 mm, although other diameters are contemplated.

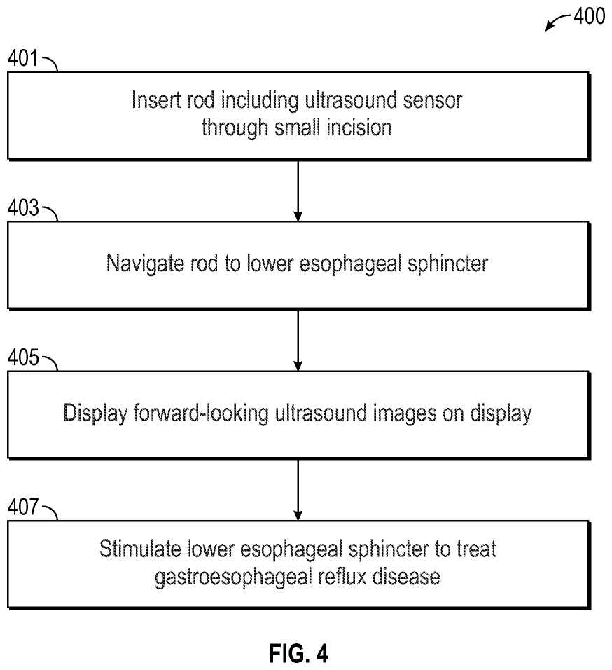

FIG. 4 illustrates a flowchart for a method for treating gastroesophageal reflux disease, and is described as method 400. Some or all of the steps of method 400 may be carried out manually or by components of a computing device or a surgical robotic system. Method 400 begins at step 401 where an elongated rod 134 including an ultrasound sensor 140 is inserted through an abdominal incision. The elongated rod 134 may be inserted with the cannula 133 around the elongated rod 134. The cannula 133 provides access to the lower esophageal sphincter after the elongated rod 134 is removed from the cannula 133. In step 403, the elongated rod 134 is navigated to a lower esophageal sphincter. In step 405, images generated by the ultrasound sensor 140 are displayed on a display 110 for viewing the anatomy in front of the elongated rod 134.

In step 406, the stimulation electrode 300 is implanted adjacent to the lower esophageal sphincter. As noted above, if the stimulation electrode 300 includes a helical tip 305 at its distal end, step 406 may also include screwing the helical tip 305 of the stimulation electrode 300 into the lower esophageal sphincter, for example using the elongated rod 134 or another insertion tool, or by rotating the stimulation electrode 300 itself, for example, at a proximal end of the stimulation electrode 300. In step 407, the lower esophageal sphincter is stimulated to treat gastroesophageal reflux disease via the stimulation electrode 300.

Additionally, method 400 may include a step of reducing recurrent or maximum stress on the conductive portion of the stimulation electrode 300 by guiding the stimulation electrode 300 through connective or striated muscle tissue in order to support the stimulation electrode 300.

Although not illustrated, the system 10 may also include transesophageal ultrasound to provide global guidance, in addition to, or in place of either or both of the ultrasound sensor 140 and the electromagnetic tracking system, which may be beneficial in electrode implant location selection. Implant locations can be electrically stimulated through transesophageal access, where appropriate sphincter response can be directly confirmed. In aspects, the elongated rod 134 or an electromagnetic marker electrode attached to the elongated rod 134 (e.g., stimulation electrode 300) may provide a target for an abdominal placement of elongated rod 134.

In accordance with the aspects described above, the target location of the lower esophageal sphincter may be identified from endoluminal ultrasound and/or preoperative imaging (e.g., CT imaging). In aspects, the stimulation electrode carrier, for example, the elongated rod 134 or another placement tool, may be steered to the target location (e.g., lower esophageal sphincter) by using ultrasound as a beacon against another ultrasound source, by three-dimensional navigation coordinating the location of the two instalments (e.g., one instrument in the esophagus and another instrument tunneling a lead placement device). Additionally, or alternatively, preoperative imaging may be registered to the location of the implantation tool 130 with the preoperative images being mapped into a three-dimensional model.

Although the method and system described above is described as being navigable to the lower esophageal sphincter, it is also contemplated that the target location or the navigation described is on a crus muscle.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system comprising:
an implantation tool configured to navigate through an incision to a lower esophageal sphincter muscle, the implantation tool including:
a handle configured to be grasped by a user;
a cannula extending distally from the handle and defining a lumen therethrough;
an elongated rod removably positioned through the lumen of the cannula; and
an ultrasound sensor disposed at a distal portion of the elongated rod and oriented to provide a forward-looking perspective of ultrasound images of objects distally of the elongated rod;
a stimulation electrode positionable through the cannula of the implantation tool and configured to stimulate the lower esophageal sphincter muscle to treat gastroesophageal reflux disease; and
a display configured to display ultrasound images generated from the ultrasound sensor during navigation of the implantation tool.

2. The system according to claim 1, further comprising a tracking system configured to track a position of the implantation tool.

3. The system according to claim 2, wherein the tracking system is an electromagnetic tracking system configured to generate an electromagnetic field and the implantation tool includes a position sensor configured to sense the electromagnetic field.

4. The system according to claim 1, wherein the implantation tool includes a position reference guide extending from the handle and configured to provide extracorporeal positional and directional reference during navigation of the implantation tool.

5. The system according to claim 1, wherein the stimulation electrode is configured to removably secure to a lower esophageal sphincter muscle.

6. The system according to claim 1, wherein the stimulation electrode includes a helical tip at its distal portion to screw the helical tip of the stimulation electrode into the lower esophageal sphincter muscle upon rotation of the stimulation electrode.

7. The system according to claim 1, wherein the cannula is removable from the implantation tool such that the elongated rod is removable from the cannula after a distal portion of the cannula is navigated proximate a lower esophageal sphincter muscle to provide access for placement of the stimulation electrode through the cannula.

8. The system according to claim I, wherein the ultrasound sensor is a capacitive micromachined ultrasonic transducer.

9. The system according to claim 1, further comprising an ultrasound imaging device, separate from the ultrasound sensor of the implantation tool, the ultrasound imaging device configured to acquire ultrasound data externally from a patient.

10. A implantation tool configured to navigate through a small incision to a lower esophageal sphincter muscle, the implantation tool comprising:
a handle configured to be grasped by a user;
a cannula extending distally from the handle and defining a lumen therethrough;
an elongated rod removably positioned through the lumen of the cannula; and
an ultrasound sensor disposed at a distal portion of the elongated rod and oriented to provide a forward-looking perspective of ultrasound images of objects distally of the elongated rod.

11. The implantation tool according to claim 10, further comprising a position sensor configured to operably couple to a tracking system for tracking a position of the implantation tool during navigation.

12. The implantation tool according to claim 10, wherein the cannula is configured to receive a stimulation electrode configured to stimulate the lower esophageal sphincter muscle to treat gastroesophageal reflux disease through the lumen.

13. The implantation tool according to claim 12, wherein the stimulation electrode includes a helical tip at its distal portion to screw the helical tip of the stimulation electrode into the lower esophageal sphincter muscle upon rotation of the stimulation electrode.

14. The implantation tool according to claim 12, wherein the cannula is removable from the implantation tool such that the elongated rod is removable from the cannula after a distal portion of the cannula is navigated proximate a lower esophageal sphincter muscle to provide access for placement of the stimulation electrode through the cannula.

15. The implantation tool according to claim 10, wherein the ultrasound sensor is a capacitive micromachined ultrasonic transducer.

16. The implantation tool according to claim 10, further comprising a position reference guide extending from the handle and configured to provide extracorporeal positional and directional reference during navigation of the implantation tool.

17. A method for treating gastroesophageal reflux disease comprising:
inserting an elongated rod surrounded by a cannula through an abdominal incision;
navigating the elongated rod and cannula to a lower esophageal sphincter muscle;
displaying front view ultrasound images during the navigating, the front view ultrasound images generated by an ultrasound sensor coupled to a distal portion of the elongated rod;
implanting a stimulation electrode into the lower esophageal sphincter muscle; and
stimulating the lower esophageal sphincter muscle by the stimulation electrode to treat the gastroesophageal reflux disease.

18. The method according to claim 17, further comprising utilizing an electromagnetic tracking system to track a position of the elongated rod during the navigating.

19. The method according to claim 17, further comprising screwing a helical tip of the stimulation electrode into the lower esophageal sphincter muscle by rotating the stimulation electrode prior to stimulating the lower esophageal sphincter muscle.

20. The method according to claim 17, further comprising displaying secondary ultrasound data during the navigating of the elongated rod.
